(19) United States
(12) Patent Application Publication
(10) Pub. No.: US 2021/0322223 A1
(43) Pub. Date: Oct. 21, 2021

(54) FIXATION METHODS FOR DEVICES IN TUBULAR STRUCTURES

(71) Applicant: Staton Techiya LLC, Lighthouse Point, FL (US)

(72) Inventors: Steven Wayne Goldstein, Delray Beach, FL (US); John P. Keady, Fairfax Station, VA (US)

(73) Assignee: Staton Techiya LLC, Lighthouse Point, FL (US)

(21) Appl. No.: 17/364,866

(22) Filed: Jun. 30, 2021

Related U.S. Application Data
(63) Continuation-in-part of application No. 14/956,365, filed on Dec. 1, 2015.
(60) Provisional application No. 62/086,019, filed on Dec. 1, 2014.

(51) Int. Cl. A61F 11/08; A61B 5/00

(52) U.S. Cl. CPC A61F 11/08; A61B 5/6882; A61F 2011/085; A61B 5/6817; A61B 2562/0204; A61B 5/6862

(57) ABSTRACT

A method and system of traversing a device through an accommodating conduit to reach a target area within the accommodating conduit can include varying an amount of elasticity of the device or an amount of torsion moment of the device applied to the accommodating conduit or varying both to minimize bending forces as the device traverses the accommodating conduit. The method or system upon reaching the target area within the accommodating conduit, can further include varying the elasticity or the torsion moment of the device or varying both to cause the device to stiffen. Other embodiments are disclosed.

### CROSS REFERENCE TO RELATED APPLICATIONS

This Application is a continuation in part of and claims priority to U.S. patent application Ser. No. 14/956,365, filed 1 Dec. 2015, which claims the priority benefit of Provisional Application No. 62/086,019 filed on Dec. 1, 2014, the entire contents and disclosure of which are incorporated herein by reference.

### FIELD

The present disclosure pertains to fixation methods and devices within tubular structures, and more particularly, to fixation methods and devices for miniature medical devices in tubular structures.

### BACKGROUND

Rapid miniaturization of medical devices is about to revolutionize the whole medical field. Smart devices are currently being designed for insertion into the human body for diagnostics, monitoring, drug delivery and in the future for treatment, quality of life enhancements and even surgery. At the same time, certain new challenges need to be overcome to make those devices safe and effective. One of the first problems that occur is a stable fixation of the device inside human body. Implantable devices are often permanently attached to the anatomical structures and cannot be easily removed or repositioned. This approach is not applicable when the device needs to travel inside the body or be inserted with minimal surgical impact. One example of a device that travels inside the body that does not necessarily require fixation is the Given Imaging PillCam®, that traverses the digestive system and transmits real time video to the operator. Another example of a device that does not necessarily require fixation is a device that is frequently inserted and released as with in-the-ear devices.

Tubular structures are very common in human body. Current solutions (i.e. cardio-vascular stents) apply radial forces on the walls to increase friction with tissue and avoid mutual motion. With this approach, however, the tissue is under perpetual pressure, which is usually not desirable. Tissue necrosis is often experienced from long-term radial force within a biological lumen.

A method and device that mitigates or avoids most or all of the issues described above is unknown.

### SUMMARY

Embodiments in accordance with the present disclosure provides a fixation method and fixation devices for miniature medical devices in tubular structures.

A novel approach to stable fixation of miniature devices in tubular structures can include a key idea to utilize curvature or torsion or both that often naturally occur in organic shapes due to certain tissue growth mechanisms. [See Thompson, “On Growth and Form”]. Intuitively speaking, torsion is the amount of axial rotation that occurs in the tubular structure and curvature is the amount of bending. In order to provide a more formal definition, the concept of a moving frame is introduced herein.

With respect to a moving frame with reference to FIG. 1A, a general tubular shape 10 can be described in terms of its axial curve A(s), where s is the arc-length, and the generating cross-section contour c. The resulting structure can be conveniently described by associating a moving frame E(s)(e₁, e₂, e₃) to each point on the axial curve A (see again, FIG. 1A). Then, the curvature κ(s) and torsion τ(s) of the tubular shape 10 can be evaluated as angles of rotation of the moving frame. A Frenet frame is most frequently used in differential geometry [See DoCarmo, “Differential Geometry”]. This frame is defined by tangent T(s), normal N(s) and bi-normal B(s) of the axial curve.

Thus, curvature and torsion can be evaluated using Frenet-Serret formulae as follows:

dT/ds=κN

dN/ds=−κT+τB

dB/ds=−τN

While having some advantages, Frenet frame is undefined in regions of unstable second derivative. Therefore, in many practical applications minimal rotation frame (MRF) is more appropriate. Unlike with Frenet frame, MRF cannot be defined locally. It is rather defined globally by minimizing an amount of rotation between boundary conditions defined on A. In other words, it provides the transformation of the frame from the initial to final position with minimal rotation around A.

Curvature and torsion are intrinsic properties of the curve and therefore invariant under Euclidean transformations. Consequently, those properties are independent of the reference system of coordinates. It is also known that any curve in space can be uniquely identified by its curvature K(s) and torsion T(s) defined on the curve [DoCarmo, Differential Geometry]. Or more formally, two curves are congruent if and only if their K(s) and T(s) are identical. In practice, if the curves are smooth, it is sufficient to require identity of intrinsic properties in a limited number of points in order to guarantee congruency. This observation provides the basis for the proposed fixation methods described in the following section.

The proposed fixation methods are based on forces of friction and elasticity. The idea is inspired by locomotion of organisms such as worms and snakes inside narrow tunnels. In this invention, three different methods are proposed. Each method is suitable for certain combination of the intrinsic properties of the tubular structure introduced above. The following table summarizes all possible combinations of the intrinsic properties.

| Method | Curvature | Torsion |
|---|---|---|
| 1 | Yes | No |
| 2 | No | Yes |
| 3 | Yes | Yes |

### BRIEF DESCRIPTION OF THE DRAWINGS

FIG. 1A is a pictorial diagram of a moving frame in accordance with an exemplary embodiment;

FIG. 1B is a pictorial diagram of a tubular structure exhibiting bending following a curvature profile in accordance with an exemplary embodiment;

FIG. 1C is a pictorial diagram of a tubular structure exhibiting rotation around an axial curve that produces torsion in accordance with an exemplary embodiment;

FIG. 2 is a pictorial diagram of an elliptical balloon in an ear canal;

FIG. 3 is an ear canal cross section rotation in accordance with an embodiment;

FIG. 4 is a flowchart for a fixation method in accordance with an exemplary embodiment;

FIG. 5 is a block diagram of a system in accordance with an embodiment herein;

FIG. 6A, FIG. 6B, and FIG. 6C illustrates the insertion of a controllable flexible stent;

FIG. 7A illustrates a stent including expanding and contracting elements;

FIG. 7B and FIG. 7C illustrate at least one exemplary embodiment of fabricating expansion and contracting elements;

FIG. 7D illustrates a flexible stent bending by expanding and contracting elements;

FIG. 8A, FIG. 8B, and FIG. 8C illustrate a two section flexible stent using expanding and contracting elements to set the stent in a controlled shape;

FIG. 9A and FIG. 9B, and FIG. 10A, and FIG. 10B illustrate sections of a flexible stent;

FIG. 11 illustrates a side view of a flexible stent;

FIG. 12 illustrates an earphones;

FIG. 13 and FIG. 14 illustrate exploded views of one embodiment of an earphone;

FIG. 15 is a schematic diagram of a system for utilizing earphones according to an embodiment of the present disclosure;

FIG. 16 is a schematic diagram of a machine in the form of a computer system which a set of instructions, when executed, may cause the machine to perform any one or more of the methodologies or operations of the systems and methods for utilizing an earphone according to embodiments of the present disclosure;

FIG. 17 is a schematic exploded view of a multimicrophone earphone;

FIG. 18 illustrates the earphone of FIG. 17 with components inserted;

FIG. 19 illustrates left and right earphones prior to insertion of a user's ears; and

FIG. 20 illustrates the internal circuitry of an earphone.

### DETAILED DESCRIPTION

Methods and devices in accordance with the embodiments are not limited to exact methods disclosed and variations within contemplation of the embodiments can certainly include variations that include one or more methods in combination and steps in methods that are skipped, or completed in different order or additional steps beyond the steps disclosed herein.

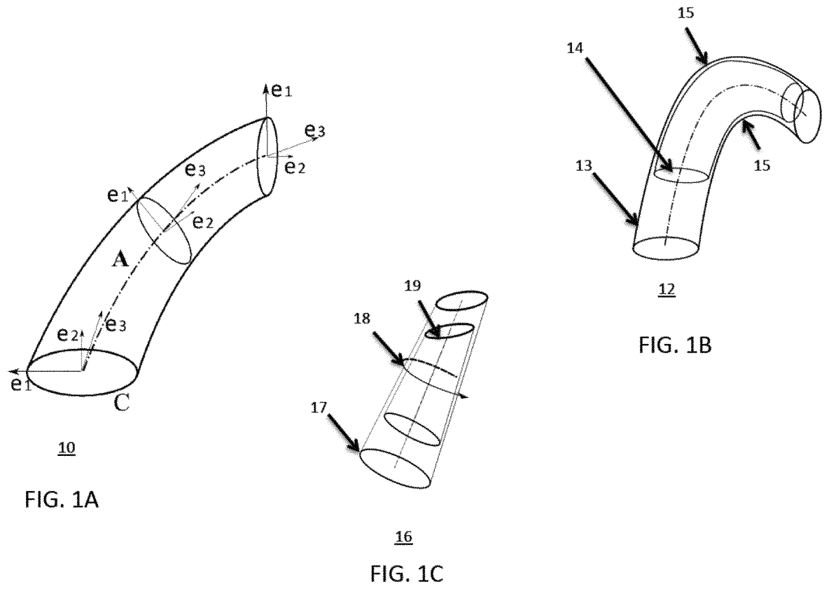

Method #1. When a tubular structure 13 is bending at a bend 15 as illustrated in a system 12 of FIG. 1B, it is proposed in some embodiments to vary the elasticity of a device 14 in order to achieve fixation. When the device 14 is traveling through the accommodating structure 13, the device 14 can be very flexible and can be configured to not apply any bending forces on the structure 13. Once the device 14 will arrive to the desired location, the device can be configured to become stiff and follow the curvature profile (15) of the tubular structure. This effect can be achieved for example by using mechanical interlocking or electro-magnetic forces, Electro activated polymers, or with the introduction of gas or fluid into the tubular structure with an additional ability to regulate the operating pressure of the fluid in the tubular structure. As a result, the device 14 will be immobilized in its current position and shape within the biological lumen at a desired location. Stability of the device 14 may be controlled through the amount of bending force applied on the accommodating structure 14.

FIG. 1B illustrates a method of traversing a device 14 through an accommodating conduit (such a biological lumen) 13 to reach a target area within the accommodating conduit. In one example, the target area can coincide with a bend 15 in the structure 13. In some embodiments, the method can include varying an elasticity of the device to minimize bending forces as the device traverses the accommodating conduit and upon reaching the target area within the accommodating conduit, further varying the elasticity of the device to cause the device to stiffen. In some embodiments the method further follows a curvature profile of the accommodating conduit at a target area when the device is caused to stiffen. As noted above, varying the elasticity of the device can be done by any number of techniques, for example, using mechanical interlocking forces, using electro-magnetic forces, using Electro activated polymers (EAPs), or by introducing one of gas or fluid into the device with an additional ability to regulate the operating pressure of the fluid in the device.

In some embodiments, the device 14 can be immobilized in its current position and shape within the accommodating conduit 13 at the target area. Additionally, a stability of the device is controlled through an amount of bending force applied on the accommodating conduit.

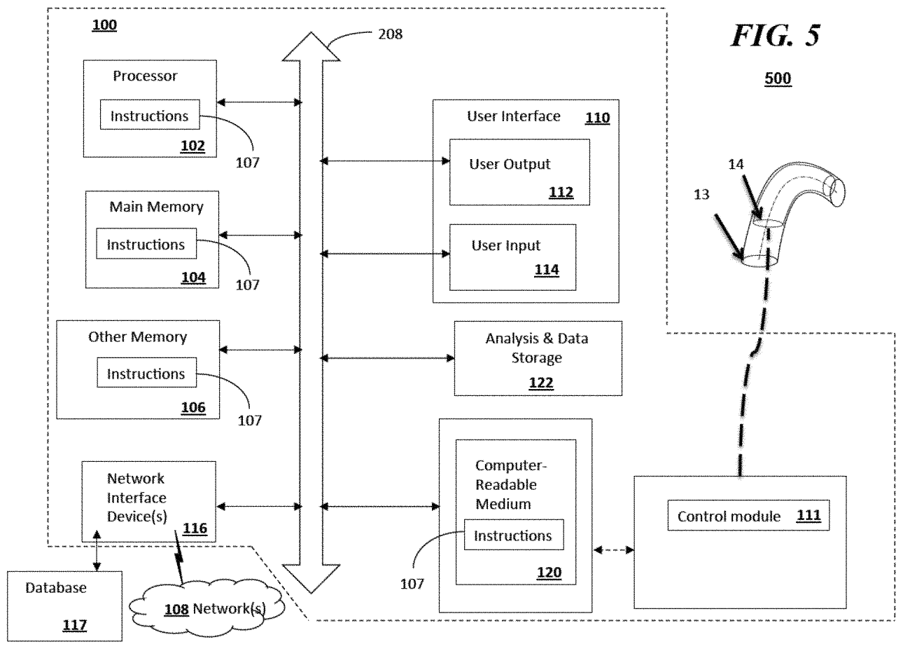

A device 14 in some embodiments can include one or more members of the device having a controllable elasticity where the device is configured to traverse an accommodating conduit 13 (such as a biological lumen, e.g., artery, vein, etc.). The device 14 can include or be operatively coupled to a controller or processor (such as processor 102 or control module 111 of FIG. 5) for controlling the controllable elasticity of the device as the device traverses the accommodating conduit towards a target area of the accommodating conduit and for further causing one or more members of the device to selectively stiffen as the device reaches the target area. As noted above, such a device can include mechanical interlocking members that apply mechanical interlocking forces, or use electro-magnetic members that applying electro-magnetic forces, or use Electro activated polymers (EAPs). In some embodiments, the controller controls the introduction of one of gas or fluid into the device with an additional ability to regulate the operating pressure of the fluid in the device.

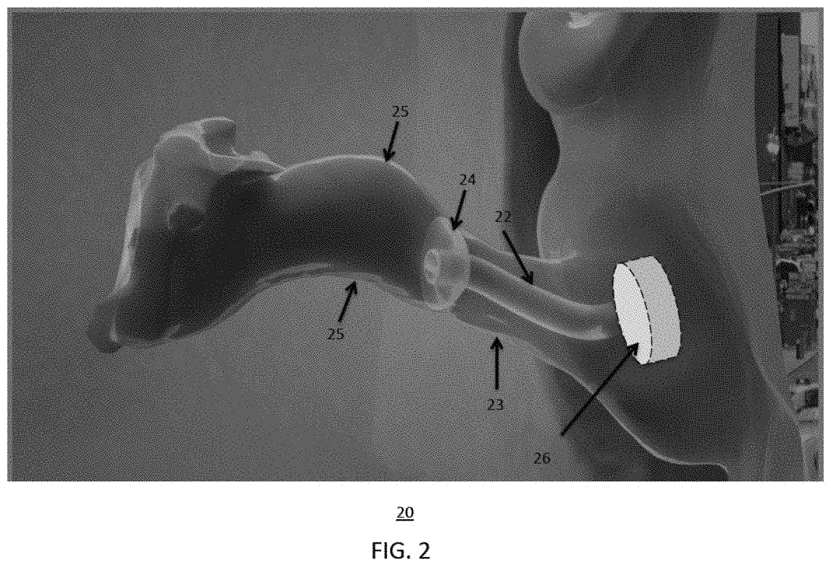

Method #2. When a tubular structure 17 is rotating around its axial curve producing torsion, fixation can be achieved by utilizing torsional moment 18 as illustrated in system 16 of FIG. 1C. Similar to Method #1, a device 19 can be very flexible during insertion. Once in place, the device 19 will constrain two or more cross-sections in their relative orientation, following the torsion of the tubular structure. This can be done using inflatable balloons (see balloon 24 of FIG. 2) which are created to fit within the geometry of the cross section, or by extending supports into walls of the tubular structure. With this method, stability of the device can be controlled by varying the amount of torsion moment applied on the accommodating structure. The balloons are to be adhered, molded or otherwise affixed to the tubular structure in alignment or in misalignment based on the amount of torsion and ultimate fixation required.

In some embodiments and referring again to FIG. 1C, a method of traversing a device 19 through an accommodating conduit 17 (such a biological lumen) to reach a target area within the accommodating conduit can include the steps of varying an amount of torsion moment of the device 19 applied to the accommodating conduit 17 to minimize bending forces as the device traverses the accommodating conduit 17 and upon reaching the target area within the accommodating conduit, further varying the torsion moment of the device 19 to cause the device 19 to stiffen. In some embodiments, varying the amount of torsion moment of the device applied to the accommodating conduit can be done by using inflatable balloons configured to fit within a geometry of a cross section of the accommodating conduit. In some embodiments, varying the amount of torsion moment of the device applied to the accommodating conduit can alternatively be done by using support members that extend into the walls of a tubular structure of the device. A stability of the device can also be controlled through an amount of torsion moment applied on the accommodating conduit.

A device 19 in some embodiments can include one or more members of the device having a controllable torsion moment of the device where the device is configured to traverse an accommodating conduit 17 (such as a biological lumen, e.g., artery, vein, etc.). The device 19 can include or be operatively coupled to a controller or processor (such as processor 102 or control module 111 of FIG. 5) for controlling the controllable torsion moment of the device as the device traverses the accommodating conduit 17 towards a target area of the accommodating conduit 17 and for further causing one or more members of the device to selectively stiffen as the device reaches the target area. In some embodiments, the controllable torsion moment of the device 19 is controlled by using one or more inflatable balloons (see balloon 24 of FIG. 2) configured to fit within a geometry of a cross section of the accommodating conduit. In some embodiments, the one or more inflatable balloons can be affixed to a tubular structure of the device in alignment or in misalignment with the accommodating conduit based on the amount of torsion and ultimate fixation desired at the target area. In some embodiments, the controllable torsion moment of the device is controlled by using support members that extend into the walls of a tubular structure of the device.

Method #3. When the tubular structure of interest has a helical shape, where both curvature and torsion occur, it is possible to utilize both fixation methods (Method #1 and Method #2) described above simultaneously. In such an instance, which often occurs in human body, increased fixation can be achieved. One particular example of such structure, the external auditory canal (EAC), is discussed in detail further below. However, a method in accordance with the embodiments is not limited to using both fixation methods simultaneously. As contemplated herein, either method can be used either alone or in a serial fashion or simultaneously as appropriately suited for a given structure and application.

Thus, in some exemplary embodiments, a method of traversing a device through an accommodating conduit (such a biological lumen) to reach a target area within the accommodating conduit can include varying an amount of elasticity of the device OR varying an amount of torsion moment of the device applied to the accommodating conduit OR varying both (elasticity and torsion moment) to minimize bending forces as the device traverses the accommodating conduit. Upon reaching the target area within the accommodating conduit, the method can further vary the elasticity OR vary the torsion moment of the device (OR vary both) to cause the device to stiffen. As note above, in some embodiments the step of varying comprises varying the amount of elasticity AND the amount of torsion moment of the device and further varying the elasticity or the torsion moment of the device (or varying both) upon reaching the target area.

A device in some embodiments can include one or more members of the device having a controllable elasticity of the device OR a controllable torsion moment of the device, where the device is configured to traverse an accommodating conduit (such as a biological lumen, e.g., artery, vein, etc.). As in other embodiments, the device can also include a controller or processor for controlling the controllable elasticity OR the controllable torsion moment of the device (or both) as the device traverses the accommodating conduit towards a target area of the accommodating conduit and for further causing one or more members of the device to selectively stiffen as the device reaches the target area.

One particular accommodating conduit that can be used as an example for fixation of devices is the fixation of devices in the external auditory canal (EAC). In recent years, completely-in-the-canal (CIC) devices became main stream in the hearing aid industry. The smaller and smarter those devices become, the wider range of possible applications opens. Currently, CIC devices are personalized for each customer, so the tight fit guarantees device stability in the ear. However, as electronic components and manufacturing technologies evolve, it is becoming possible to come up with one-fits-most designs which will be suitable for almost anyone. Consequently, new fixation techniques are developed herein to ensure reliable device positioning.

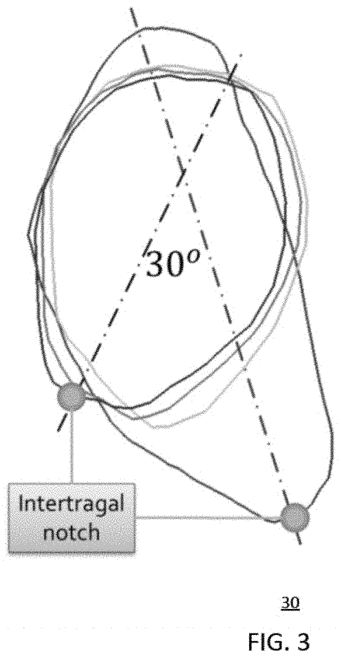

The EAC has a cross section of an elliptical shape near the orifice that gradually converges towards the tympanic membrane. The axial curve of the canal has two distinctive high curvature regions, known as 1st and 2nd bends, as can be seen in in the illustration 20 of FIG. 2. The EAC is an example of an accommodating structure 23 that has at least one bend 25. A device 24 such as a balloon attached to an insertion member or stem 22 is illustrated as the balloon traverses the accommodating structure 23. Our research has shown that the average axial frame rotation between the orifice (or opening of the ear) and the 1st bend is about 30 degrees, shown in the illustration 30 of FIG. 3. The intertragal notch is illustrated on illustration 30 as a reference point. This rotation can be leveraged for secure fixation of CIC devices in the ear based on a desired insertion depth. With the proposed embodiments, the device in some embodiments can be equipped with an elliptical balloon on the canal side and stopping ring or a flange or another balloon on the orifice side. During the insertion, the balloon can slightly rotate around the axis. However, once the device is inserted, the rotation angle is fixed and due to the difference between actual canal angle of rotation and the one imposed by the device, a moment will be applied by the tissue on the balloon. This moment will prevent the device from accidentally escaping out of the ear canal. The fixed angle of rotation can be modified by the wearing person once the device is inserted in order to increase or decrease the locking moment.

In addition, curvature of the ear canal can be utilized to provide even more secure fixation. In order to achieve such greater secure fixation, the device should have a flexible body. After insertion, the shape of the device will adapt to the accommodating ear and fix. Similar to the rotation, bending of the device can be controlled by the user to maximize comfort and reliability.

In a some embodiments as illustrated in FIG. 2 where a fixation device 24 is configured for placement in an external auditory canal 23, such fixation device can include an elliptical shaped balloon on a distal end of an insertion member 22. In some embodiments the fixation device 24 is a flexible body member. In some embodiments, the fixation device 24 can include a flange or stopping ring or another balloon 26 on a proximal end of the insertion member 22. During insertion of the fixation device in the EAC, the elliptical shaped balloon rotates around an axial curve of the EAC and a rotation angle of the balloon is fixed at an angle defined by a difference between an actual canal angle of rotation and an angle imposed by the fixation device causing a locking moment applied by a tissue of the EAC on the balloon. In some embodiments, the locking moment is further modified by a user of the fixation device.

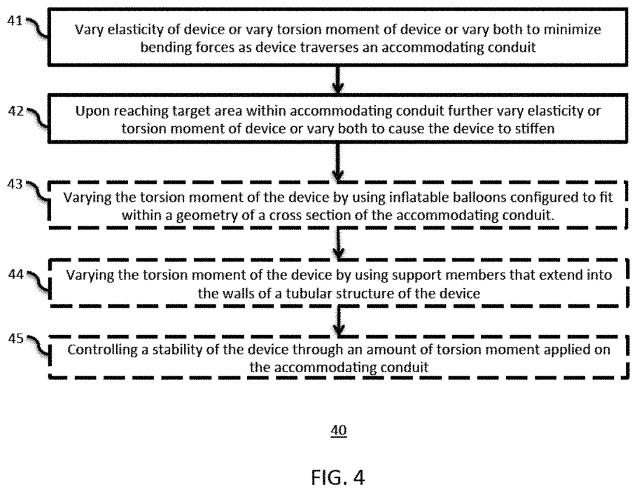

Referring to FIG. 4, a method 40 of traversing a device through an accommodating conduit to reach a target area within the accommodating conduit can include the step 41 of varying an amount of elasticity of the device or an amount of torsion moment of the device applied to the accommodating conduit or varying both to minimize bending forces as the device traverses the accommodating conduit and the step 42 of further varying the elasticity or the torsion moment of the device or varying both to cause the device to stiffen upon reaching the target area within the accommodating conduit. The embodiments can encompass variations as exemplified by the claims. In some embodiments, the method 40 can vary the torsion moment of the device by using at least one inflatable balloon configured to fit within a geometry of a cross section of an accommodating conduit as shown at step 43. In some embodiments, the method 40 can vary the torsion moment of the device by using support members that extend into the walls of a tubular structure of the device as in step 44. In some embodiments, the method 40 can include the step 45 of controlling a stability of the device through an amount of torsion moment applied on the accommodating conduit.

Referring FIG. 5, an information processing system 100 or other system 500 can be communicatively coupled with a module 111 for controlling elasticity or the torsion moment of a device 14 as it traverses a structure 13 and subsequently is put in a target area within the structure 13 as described in the aforementioned methods above. According to this example, at least one processor 102, responsive to executing instructions 107, performs operations to communicate with the module 111 via a bus architecture 208, as shown. The at least one processor is communicatively coupled with main memory 104 or other memory 106, and a computer readable medium 120. The processor is communicatively coupled with an Analysis & Data Storage 122 that, according to various implementations, can maintain stored information used by, for example, the module 111 and more generally used by the information processing system. Additionally, according to another example, an history or repository of shapes or structures can be maintained or stored in the Analysis & Data Storage. The module 111, and the information processing system 100, can use the information from the storage 122.

The information processing system includes a user interface 110 that comprises a user output interface 112 and user input interface 114. Examples of elements of the user output interface can include a display, a speaker, one or more indicator lights, one or more transducers that generate audible indicators, and a haptic signal generator. Examples of elements of the user input interface can include a keyboard, a keypad, a mouse, a track pad, a touch pad, a microphone that receives audio signals. The received audio signals, for example, can be converted to electronic digital representation and stored in memory, and optionally can be used with voice recognition software executed by the processor to receive user input data and commands.

A network interface device 116 is communicatively coupled with the processor 102 and provides a communication interface for the information processing system to communicate via one or more networks 108. The networks can include wired and wireless networks, and can be any of local area networks, wide area networks, or a combination of such networks. For example, wide area networks including the internet and the web can inter-communicate the information processing system with other one or more information processing systems that may be locally, or remotely, located relative to the information processing system. It should be noted that mobile communications devices, such as mobile phones, Smart phones, tablet computers, phablets, lap top computers, and the like, which are capable of at least one of wired and/or wireless communication, are also examples of information processing systems within the scope of the present disclosure. The network interface device 116 can provide a communication interface for the information processing system to access the database 117 according to various embodiments of the disclosure.

The instructions, according to the present example, include instructions for performing the functions described with respect to the flow chart of FIG. 4 or other methods described herein. It should be noted that any portion of the instructions can be stored in a centralized information processing system or can be stored in a distributed information processing system, i.e., with portions of the system distributed and communicatively coupled together over one or more communication links or networks.

The present invention may be a system, a method, and/or a computer program product. The computer program product may include a computer readable storage medium (or media) having computer readable program instructions thereon for causing a processor to carry out aspects of the present invention.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer readable program instructions described herein can be downloaded to respective computing/processing devices from a computer readable storage medium or to an external computer or external storage device via a network or networks, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described herein with reference to flowchart illustrations and/or block diagrams of methods, apparatus (systems), and computer program products according to embodiments of the invention. It will be understood that each block of the flowchart illustrations and/or block functional diagrams, and combinations of blocks in the flowchart illustrations and/or block functional diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or functional block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the computer readable storage medium is shown in an example embodiment to be a single medium, the term “computer readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium that is capable of storing or encoding a set of instructions for execution by the machine and that cause the machine to perform any one or more of the methods of the subject disclosure.

The term “computer-readable storage medium” shall accordingly be taken to include, but not be limited to: solid-state memories such as a memory card or other package that houses one or more read-only (non-volatile) memories, random access memories, or other re-writable (volatile) memories, a magneto-optical or optical medium such as a disk or tape, or other tangible media which can be used to store information. Accordingly, the disclosure is considered to include any one or more of a computer-readable storage medium, as listed herein and including art-recognized equivalents and successor media, in which the software implementations herein are stored.

Although the present specification may describe components and functions implemented in the embodiments with reference to particular standards and protocols, the disclosure is not limited to such standards and protocols. Each of the standards represents examples of the state of the art. Such standards are from time-to-time superseded by faster or more efficient equivalents having essentially the same functions.

The illustrations of examples described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Although specific embodiments have been illustrated and described herein, it should be appreciated that any arrangement calculated to achieve the same purpose may be substituted for the specific embodiments shown. The examples herein are intended to cover any and all adaptations or variations of various embodiments. Combinations of the above embodiments, and other embodiments not specifically described herein, are contemplated herein.

The Abstract is provided with the understanding that it is not intended be used to interpret or limit the scope or meaning of the claims. In addition, in the foregoing Detailed Description, various features are grouped together in a single example embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

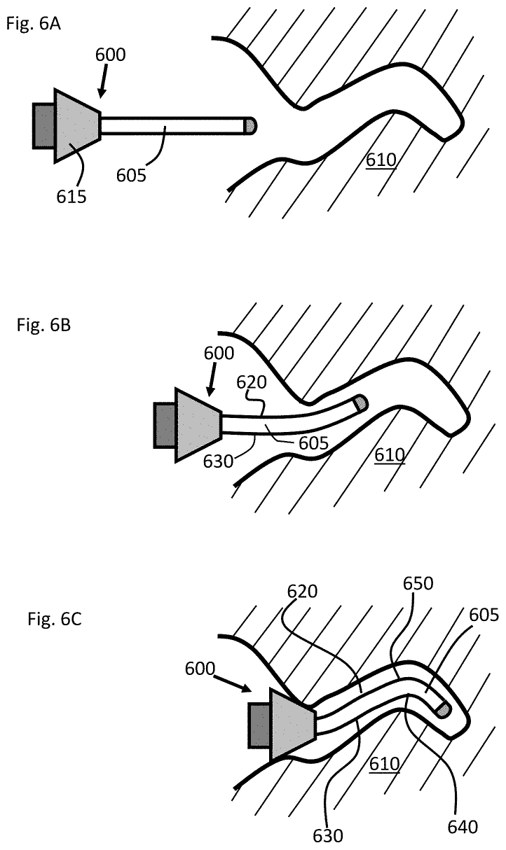

FIG. 6A, FIG. 6B, and FIG. 6C illustrates the insertion of a controllable flexible stent 605 device 600. An optional stop flange 615 controls the insertion depth into the tubular shape defined by walls 610. FIG. 6B illustrates an upward bend in the channel that is navigated by contraction 620 and expansion 630 on either side of stent 605. FIG. 6C illustrates continued insertion where further curves are maneuvered by contraction 640 and expansion 650.

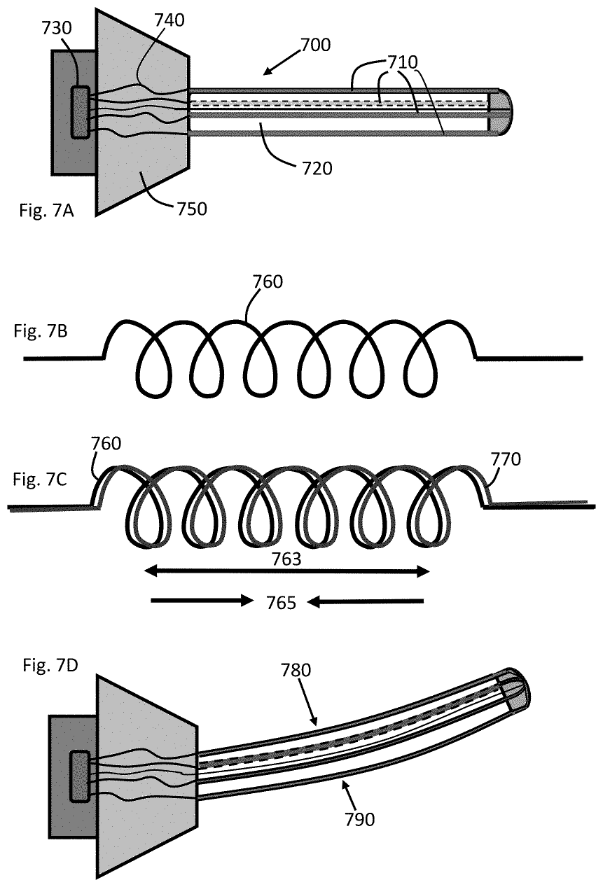

FIG. 7A illustrates a stent including expanding and contracting elements. Various expanding and contracting elements can be embedded in the stent. For example “Artificial Muscles from Fishing Line and Sewing Thread”, Carter S. Haines et al., Science 343, 868 (2014), incorporated by reference in it's entirety, describes constructing expanding/contracting elements. Note that other electroactive polymers, expanding contracting gels, can be used and the description herein is only a non-limiting example. A flexible stent 720 device 700, can include a processor 730, which can control the expanding and contracting elements 710, by electrical connections 740, by varying voltage and or current to conductive interweaved twisted conductive strands (e.g., silver coated nylon thread), which add heating locally. The device 700 can include an optional stop flange 750 that controls insertion depth by having a dimension larger than an inner diameter of a tubular opening.

FIG. 7B and FIG. 7C illustrate at least one exemplary embodiment of fabricating expansion and contracting elements. For example, a monofilament nylon can be twisted under load, then heat set (e.g., 90 C to 400 C) then cooled to room temperature. For example the filament 760 can be various materials, for example Nylon 6 monofilament fishing line, Nylon 6,6 monofilament sewing thread, Nylon 6,6 silver-plated multifilament sewing thread, polyethylene braided fishing line, and other like polymer materials. To operate the expansion 763 and contraction 765, ohm heating using a twisted conductive thread 770 can be used. Embedded expansion/contraction elements can be embedded in the stent to bend (FIG. 7D) the stent using contraction 780 and expansion 790.

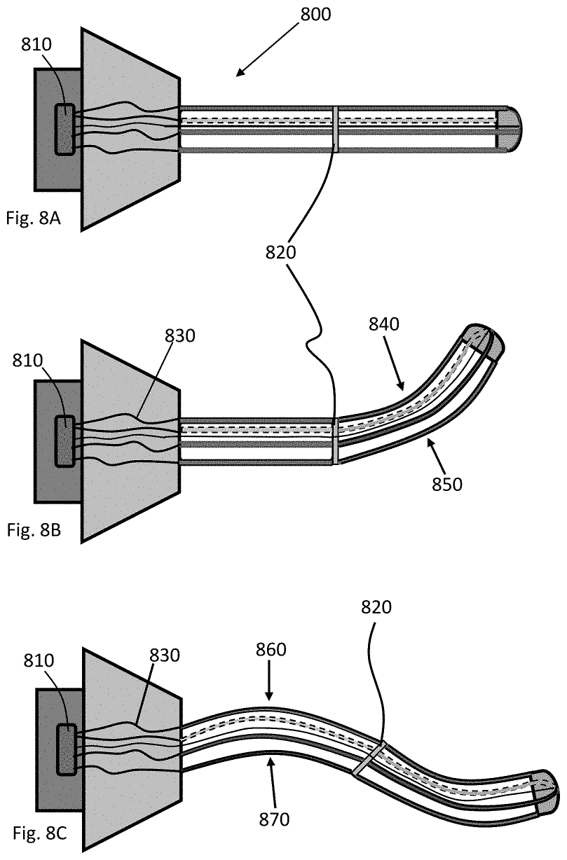

FIG. 8A, FIG. 8B, and FIG. 8C illustrate a two section flexible stent using expanding and contracting elements to set the stent in a controlled shape. The embodiments shown in FIGS. 8A, 8B and 8C show a two part flexible system 800 with a separating flexible circuit board 820. Multiple curves can be accomplished by multiple expansions (e.g., 850, 860) and multiple contractions (e.g., 840, 870) controlled by processor 810, where the expansion/contraction elements are controlled by the processor 810 connected by wires 830.

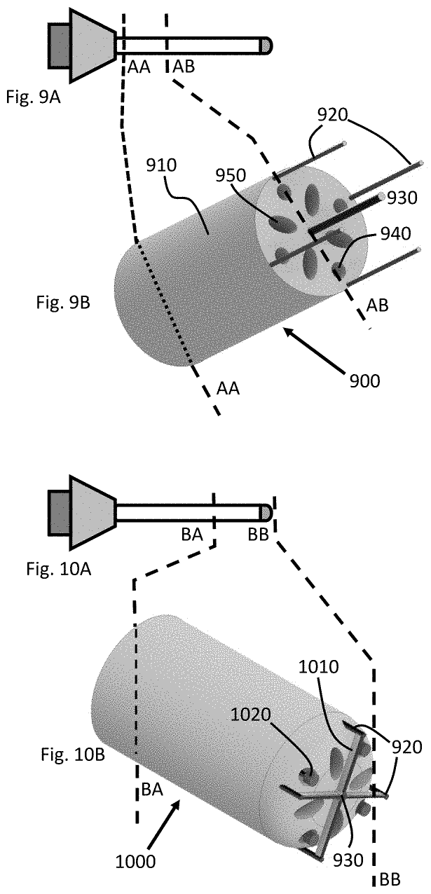

FIG. 9A and FIG. 9B, and FIG. 10A, and FIG. 10B illustrate sections of a flexible stent. FIG. 9A and FIG. 9B illustrate a mid section 900 AA-AB while FIG. 10A and FIG. 10B illustrate the tip section 1000 BA-BB. The mid section 900 of stent 910 can include embedded expansion/contraction elements 920, and optional core wire 930, which can be used to vary current and/or voltage across individual expansion/contraction elements 920. Channels 940 and 950 can be used to pass acoustic waves and/or sensor elements, for example connected to microphones, speakers, used to carry sensor wires and/or sensors. To isolate individual expansion/contraction elements 920, at the tip (FIG. 10A) a core wire 930 can carry a current or apply a voltage, connected 1010 to each expansion/contraction element, controlled by processor 810. Sensors 1020, which can be connected to processor 810 through channel 950, can monitor the stents 910 progress through tubular structures. For example the sensors 1020 can be infrared sensors or ultrasonic sensors, where each sensor monitors the closeness of the tip to the tubular structure wall. The sensor 1020 that is closest to the wall can identify which expansion/contraction element 920 to expand or contract to move the sensor away from the tubular wall. For example when sensors report equal values, within thresholds, then the stent can be considered equally positioned within the tubular structure, and the expansion/contraction of expansion/contraction elements 920 maintained.

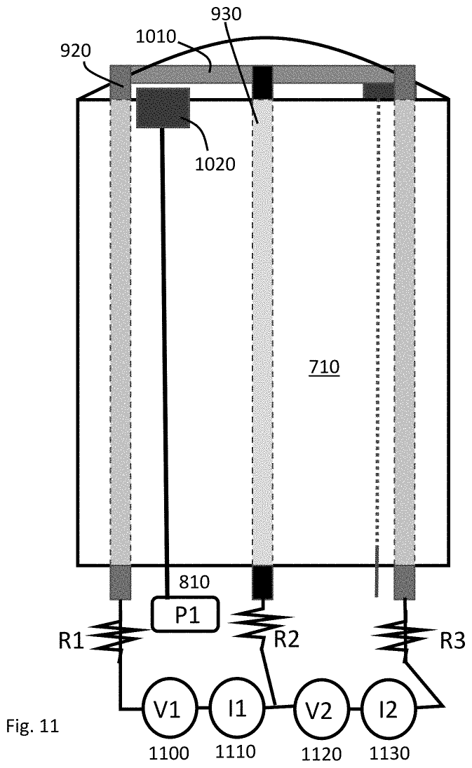

FIG. 11 illustrates a side view of a flexible stent 710, with core wire 930 connected 1010, expansion/contraction elements 920, and sensors 1020. Voltages and currents between expansion/contraction elements 920 can be controlled using resistors R1, R2, and R3, voltage power controllers V1 (1100), V2 (1120), and current controllers I1 (1110) and I2 (1130), managed by processor P1 810.

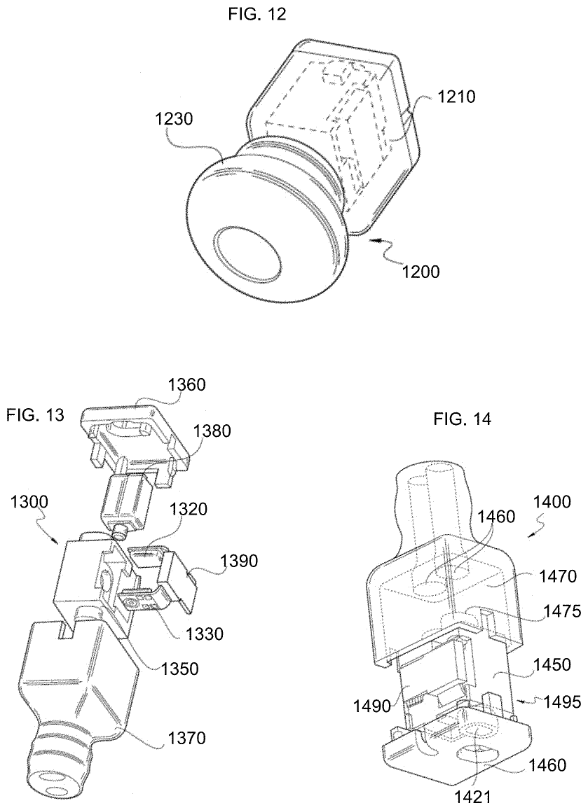

FIG. 12 illustrates an earphone 1200. The earphone 1200 shows and earphone housing (EH) 1210 that can accommodate a commercially available eartip 1230 (e.g., Comply Tips, flange tips). The earphone housing (e.g. 1210, 1470) can additionally accommodate specialized eartips (e.g. 1230). The EH 1470 can be fabricated (e.g., molded or 3D printed) from various materials (e.g., silicone, 3D printed material, metal, wood) and any material listed herein for any part of an earphone (housing, microphone, speaker, eartips) should not be interpreted as (imitative, but as examples only.

Processes, techniques, apparatus, and materials as known by one of ordinary skill in the art may not be discussed in detail but are intended to be part of the enabling description where appropriate. For example, specific materials may not be listed for achieving each of the targeted properties discussed, however one of ordinary skill would be able, without undo experimentation, to determine the materials needed given the enabling disclosure herein. For example Elastosil™ 30A, 70A, High Strength 1, 2, 3, Moldmaking Rubber (Alumilite™ products), flexible 3D printable material, silicon, urethane, rubber, however any material that can be used within the ear canal can be used for forming the shell that is inserted into the ear canal and any material that can be used for earphones (silicon, urethane, rubber, plastic, Elastosil, metal, wood, and the like) can be used in the Housing that sits in the concha. Various material can also be printed and any other materials, as mentioned if molded.

FIG. 13 illustrates an additional exploded view of a hearbud housing device 1100 with various components labelled and which are configured to be housed within the hearbud housing device 1300. For example, in certain embodiments, the components of the earphone device can include the hearbud housing device 1300, an earphone housing 1370, a cap 1360, and an electronic package housing 1350, which houses the electronics package (EP) 1390 that can include a speaker (SPKR or ECR) 1380, ambient sound microphone (ASM) 1320, an ear canal microphone (ECM) 1330, and supporting electronics that may form a part of the EP 1390. Note that any microphone that can be used in an earphone can be used for the ASM 1320 and ECM 1330. Additionally, any speaker that can be used in earphones can be used for the SPKR 1380 in the earphone device 1300.

FIG. 14 illustrates how parts fit within an earphone housing 1470 of hearbud housing device 1400 of an earphone device. A set of keys 1460 (e.g., recessed or raise keys) in the earphone housing 1470 allow the earphone housing 1470 to connect with nozzles 1475 of the EPH 1450. Thus, the electronics packaging unit (EPU) 1495 can be standardized while the earphone housing 1470 design can be varied provided the keys 1460 of the earphone housing 1470 remain the same. The EPU 1495 may include the EPH 1450 that contains the EP 1490. The ASM port/nozzle 1421 connects the ambient environment to an ASM 1320 in the EPU 1495. A cap 1460 may fit over the back of the earphone housing 1470 and the back of the EPU 1495 when inserted into the earphone housing 1470 of the hearbud housing device 1400 of the earphone device.

FIG. 13 and FIG. 14 illustrate exploded views of one embodiment of an earphone (e.g. 1300 and 1400) including two microphones (e.g. 1320, 1330, e.g. Mems Digital and Analog microphones, e.g. Knowles SiSonic Microphones, model SPH0641LM4H-1, model TO-30043-000 and other microphones that can be used in earphones or phones), a speaker (e.g. 1380, e.g., Knowles model RAB-32063, model TWFK-30017-000 and other types of speakers that can be used in earphones or phones) and DSP PCB board (e.g., CSR chips, Wolfson chips, and any other DSP chip that can process audio input that can be used in earphones or phones). The earphone (e.g., 1300, 1400) includes a cap (e.g., 1360) and an earphone housing (EH) (e.g. 1370, 1470). An electronic package housing (EPH) 1450, houses the electronic parts, for example the microphones (e.g., 1320, 1330), the speakers (e.g. 1380), and the DSP PCB board. The EH 1470 and cap 1460 can change to various configuration keeping the EPH 1450 constant, facilitating testing of the EPH 1450 (with electrical components such as microphones, speakers and DSP inserted) independent of earphone configuration (e.g., shape of housing, stent 940 length).

The materials for the EPH 1450, EH 1470 and the cap 1460 can vary depending upon desired flexibility, level of hydrophobicity required, transparency, electrical isolation, RF shielding, and other properties known by one of ordinary skill in the arts of earphone design. For example, the EPH 1450, EH 1470, cap 1460 can be 3D printed for example using resins such as Formlabs™ elastic resin, tough, grey-pro resins or other 3D printing materials as known by one of ordinary skill in fabricating small parts with tolerances of at least 2 mm. Additionally, the parts can be molded such as with Elastosil®LR3004/30B, silicone, polyurethanes, rubber, Neoprene, or any other type of moldable material as known by one of ordinary skill in the arts of designing or fabricating earphone parts with tolerances of at least 2 mm. Additionally the parts (EPH, EH, cap) can be formed of wood metal and glass.

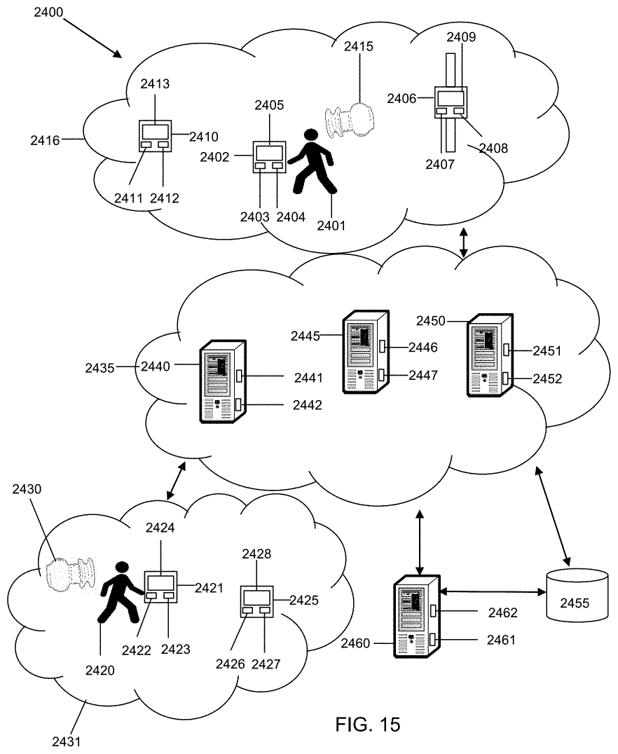

As shown in FIG. 15, a system 2400 and methods for utilizing eartips and/or earphone devices are disclosed.

The system 2400 may be configured to support, but is not limited to supporting, data and content services, audio processing applications and services, audio output and/or input applications and services, applications and services for transmitting and receiving audio content, authentication applications and services, computing applications and services, cloud computing services, internet services, satellite services, telephone services, software as a service (SaaS) applications, platform-as-a-service (PaaS) applications, gaming applications and services, social media applications and services, productivity applications and services, voice-over-internet protocol (VoIP) applications and services, speech-to-text translation applications and services, interactive voice applications and services, mobile applications and services, and any other computing applications and services. The system may include a first user 2401, who may utilize a first user device 2402 to access data, content, and applications, or to perform a variety of other tasks and functions. As an example, the first user 2401 may utilize first user device 2402 to access an application (e.g., a browser or a mobile application) executing on the first user device 2402 that may be utilized to access web pages, data, and content associated with the system 2400. In certain embodiments, the first user 2401 may be any type of user that may potentially desire to listen to audio content, such as from, but not limited to, a music playlist accessible via the first user device 2402, a telephone call that the first user 2401 is participating in, audio content occurring in an environment in proximity to the first user 2401, any other type of audio content, or a combination thereof. For example, the first user 2401 may be an individual that may be participating in a telephone call with another user, such as second user 2420.

The first user device 2402 utilized by the first user 2401 may include a memory 2403 that includes instructions, and a processor 2404 that executes the instructions from the memory 2403 to perform the various operations that are performed by the first user device 2402. In certain embodiments, the processor 2404 may be hardware, software, or a combination thereof. The first user device 2402 may also include an interface 2405 (e.g., screen, monitor, graphical user interface, etc.) that may enable the first user 2401 to interact with various applications executing on the first user device 2402, to interact with various applications executing within the system 2400, and to interact with the system 2400 itself. In certain embodiments, the first user device 2402 may include any number of transducers, such as, but not limited to, microphones, speakers, any type of audio-based transducer, any type of transducer, or a combination thereof. In certain embodiments, the first user device 2402 may be a computer, a laptop, a tablet device, a phablet, a server, a mobile device, a smartphone, a smart watch, and/or any other type of computing device. Illustratively, the first user device 2402 is shown as a mobile device in FIG. 15. The first user device 2402 may also include a global positioning system (GPS), which may include a GPS receiver and any other necessary components for enabling GPS functionality, accelerometers, gyroscopes, sensors, and any other componentry suitable for a mobile device.

In addition to using first user device 2402, the first user 2401 may also utilize and/or have access to a second user device 2406 and a third user device 2410. As with first user device 2402, the first user 2401 may utilize the second and third user devices 2406, 2410 to transmit signals to access various online services and content. The second user device 2406 may include a memory 2407 that includes instructions, and a processor 2408 that executes the instructions from the memory 2407 to perform the various operations that are performed by the second user device 2406. In certain embodiments, the processor 2408 may be hardware, software, or a combination thereof. The second user device 2406 may also include an interface 2409 that may enable the first user 2401 to interact with various applications executing on the second user device 2406 and to interact with the system 2400. In certain embodiments, the second user device 2406 may include any number of transducers, such as, but not limited to, microphones, speakers, any type of audio-based transducer, any type of transducer, or a combination thereof. In certain embodiments, the second user device 2406 may be and/or may include a computer, any type of sensor, a laptop, a set-top-box, a tablet device, a phablet, a server, a mobile device, a smartphone, a smart watch, and/or any other type of computing device. Illustratively, the second user device 2402 is shown as a smart watch device in FIG. 15.

The third user device 2410 may include a memory 2411 that includes instructions, and a processor 2412 that executes the instructions from the memory 2411 to perform the various operations that are performed by the third user device 2410. In certain embodiments, the processor 2412 may be hardware, software, or a combination thereof. The third user device 2410 may also include an interface 2413 that may enable the first user 2401 to interact with various applications executing on the second user device 2406 and to interact with the system 2400. In certain embodiments, the third user device 2410 may include any number of transducers, such as, but not limited to, microphones, speakers, any type of audio-based transducer, any type of transducer, or a combination thereof. In certain embodiments, the third user device 2410 may be and/or may include a computer, any type of sensor, a laptop, a set-top-box, a tablet device, a phablet, a server, a mobile device, a smartphone, a smart watch, and/or any other type of computing device. Illustratively, the third user device 2410 is shown as a smart watch device in FIG. 15.

The first, second, and/or third user devices 2402, 2406, 2410 may belong to and/or form a communications network 2416. In certain embodiments, the communications network 2416 may be a local, mesh, or other network that facilitates communications among the first, second, and/or third user devices 2402, 2406, 2410 and/or any other devices, programs, and/or networks of system 2400 or outside system 2400. In certain embodiments, the communications network 2416 may be formed between the first, second, and third user devices 2402, 2406, 2410 through the use of any type of wireless or other protocol and/or technology. For example, the first, second, and third user devices 2402, 2406, 2410 may communicate with one another in the communications network 2416, such as by utilizing Bluetooth Low Energy (BLE), classic Bluetooth, ZigBee, cellular, NFC, Wi-Fi, Z-Wave, ANT+, IEEE 802.15.4, IEEE 802.22, ISA100a, infrared, ISM band, RFID, UWB, Wireless HD, Wireless USB, any other protocol and/or wireless technology, satellite, fiber, or any combination thereof. Notably, the communications network 2416 may be configured to communicatively link with and/or communicate with any other network of the system 2400 and/or outside the system 2400.

The system 2400 may also include an earphone device 2415, which the first user 2401 may utilize to hear and/or audition audio content, transmit audio content, receive audio content, experience any type of content, process audio content, adjust audio content, store audio content, perform any type of operation with respect to audio content, or a combination thereof. The earphone device 2415 may be an earpiece, a hearing aid, an ear monitor, an ear terminal, a behind-the-ear device, any type of acoustic device, or a combination thereof. The earphone device 2415 may include any type of component utilized for any type of earpiece. In certain embodiments, the earphone device 2415 may include any number of ambient sound microphones that may be configured to capture and/or measure ambient sounds and/or audio content occurring in an environment that the earphone device 2415 is present in and/or is proximate to. In certain embodiments, the ambient sound microphones may be placed at a location or locations on the earphone device 2415 that are conducive to capturing and measuring ambient sounds occurring in the environment. For example, the ambient sound microphones may be positioned in proximity to a distal end (e.g. the end of the earphone device 2415 that is not inserted into the first user's 2401 ear) of the earphone device 2415 such that the ambient sound microphones are in an optimal position to capture ambient or other sounds occurring in the environment. In certain embodiments, the earphone device 2415 may include any number of ear canal microphones, which may be configured to capture and/or measure sounds occurring in an ear canal of the first user 2401 or other user wearing the earphone device 2415. In certain embodiments, the ear canal microphones may be positioned in proximity to a proximal end (e.g. the end of the earphone device 2415 that is inserted into the first user's 2401 ear) of the earphone device 2415 such that sounds occurring in the ear canal of the first user 2401 may be captured more readily.

The earphone device 2415 may also include any number of transceivers, which may be configured transmit signals to and/or receive signals from any of the devices in the system 2400. In certain embodiments, a transceiver of the earphone device 2415 may facilitate wireless connections and/or transmissions between the earphone device 2415 and any device in the system 2400, such as, but not limited to, the first user device 2402, the second user device 2406, the third user device 2410, the fourth user device 2421, the fifth user device 2425, the earphone device 2430, the servers 2440, 2445, 2450, 2460, and the database 2455. The earphone device 2415 may also include any number of memories for storing content and/or instructions, processors that execute the instructions from the memories to perform the operations for the earphone device 2415, and/or any type integrated circuit for facilitating the operation of the earphone device 2415. In certain embodiments, the processors may comprise, hardware, software, or a combination of hardware and software. The earphone device 2415 may also include one or more ear canal receivers, which may be speakers for outputting sound into the ear canal of the first user 2401. The ear canal receivers may output sounds obtained via the ear canal microphones, ambient sound microphones, any of the devices in the system 2400, from a storage device of the earphone device 2415, or any combination thereof.

The ear canal receivers, ear canal microphones, transceivers, memories, processors, integrated circuits, and/or ear canal receivers may be affixed to an electronics package that includes a flexible electronics board. The earphone device 2415 may include an electronics packaging housing that may house the ambient sound microphones, ear canal microphones, ear canal receivers (i.e. speakers), electronics supporting the functionality of the microphones and/or receivers, transceivers for receiving and/or transmitting signals, power sources (e.g. batteries and the like), any circuitry facilitating the operation of the earphone device 2415, or any combination thereof. The electronics package including the flexible electronics board may be housed within the electronics packaging housing to form an electronics packaging unit. The earphone device 2415 may further include an earphone housing, which may include receptacles, openings, and/or keyed recesses for connecting the earphone housing to the electronics packaging housing and/or the electronics package. For example, nozzles of the electronics packaging housing may be inserted into one or more keyed recesses of the earphone housing so as to connect and secure the earphone housing to the electronics packaging housing. When the earphone housing is connected to the electronics packaging housing, the combination of the earphone housing and the electronics packaging housing may form the earphone device 2415. The earphone device 2415 may further include a cap for securing the electronics packaging housing, the earphone housing, and the electronics package together to form the earphone device 2415.

In certain embodiments, the earphone device 2415 may be configured to have any number of changeable tips, which may be utilized to facilitate the insertion of the earphone device 2415 into an ear aperture of an ear of the first user 2401, secure the earphone device 2415 within the ear canal of an ear of the first user 2401, and/or to isolate sound within the ear canal of the first user 2401. The tips may be foam tips, which may be affixed onto an end of the earphone housing of the earphone device 2415, such as onto a stent and/or attachment mechanism of the earphone housing. In certain embodiments, the tips may be any type of eartip as disclosed and described in the present disclosure. The eartips as disclosed in the present disclosure may be configured to facilitate distributed reduced contact force, sound isolation for sound in the ear canal of the first user 2401 (i.e. between the ambient environment and the ear canal environment within an ear of the first user 2401), mold into a variety of forms and/or positions, encapsulate volumes upon insertion into an ear aperture of the first user 2401, have a pressure adjusting design, facilitate notched stent retention (i.e. on a stent of the earphone housing), facilitate stent insertion into an ear canal of the first user 2401 via an ear aperture of the first user 2401, or any combination thereof. In certain embodiments, the eartip may be designed to provide sound isolation capability that is at least as effective as conventional foam and/or flange tips. Notably, the eartips may be manufactured and configured to be made in any desired size specifications and/or materials, and may be tailored to each individual user, such as first user 2401. Additionally, an eartip according to the present disclosure may be made of a non-porous material that is not closed cell foam or open cell foam.

In certain embodiments, the eartip may be designed so that the earphone device's 2415 retention force on the ear canal walls of the first user 2401 may be distributed over a larger area than traditional foam or flange tips allow, thereby reducing the pressure on the ear canal walls of the first user 2401. Unlike foam tips, which primarily provide a restoring radial force that exerts pressure against the ear canal walls of a user, the eartip is designed to move both radially and axially, which allows for more give and redistribution of contact over a larger area, and, thus, decreases the retention pressure. As a result, this allows for increased comfort for the user and allows the user to utilize the eartip for an extended period of time when compared to traditional foam and/or flange tips. In certain embodiments, the eartip utilized with the earphone device 2415 may be configured to encapsulate a volume of gas and/or liquid. In either case (i.e. gas or liquid), the bulk of sound isolation provided by the eartip is achieved through the reflection of ambient sound waves so that the encapsulated volume can be low mass. In certain embodiments, portions of the eartip may encapsulate a volume with the ability to release volume when pressed upon without having to incorporate complicated valves. The encapsulated volume may be achieved by the ear canal wall pressing radially and/or axially against the outer surfaces of the eartip, which may force the outer portion of the eartip to seal with the inner portion of the eartip. In certain embodiments, the inner portion of the eartip may be small than the outer diameter of the stent of the earphone housing upon which the eartip is placed so that upon insertion of the eartip on the stent, the inner portion stretches outward to meet the outer surface of the eartip, which further facilitates the sealing of the ear canal of the first user 2401.

In certain embodiments, the stent of the eartip, over which the eartip is placed, may be designed to have a smaller diameter front end and a larger diameter middle section to promote retention of the eartip on the stent itself. In certain embodiments, a portion of the eartip may have an inner core diameter that is smaller than the stent outer diameter so that the eartip provides radial compression upon the stent so as to enhance sealing and to add friction to prevent axial slippage within the ear canal of the first user 2401. In certain embodiments, an increased mid-section inner core diameter of the eartip may be utilized (i.e. larger than the smaller inner core diameter of the eartip), which may be configured to line up with the mid-section outer diameter of the stent of the earphone housing of the earphone device 2415. This may provide axial stability for the earphone device 2415, while simultaneously preventing axial slippage from the ear canal of the first user 2401. In certain embodiments, the eartip may have an insertion end that has a funnel shape, which aids in inserting the eartip onto the stent of the earphone housing of the earphone device 2415.

In certain embodiments, the eartip has a configuration that applies minimal force against the first user's 2401 ear canal. Additionally, the eartip can seal the first user's 2401 ear canal by providing at least 15 dB of attenuation across frequency. To facilitate manufacturability, the eartip may be molded inverted, thereby allowing inexpensive mass production. Lips of the eartip may then be folded to contact ledges to for the eartip that may be utilized by the first user 2401. Sealing and comfort depend upon an accurate fit within the first user's 2401 ear canal, and, as a result, eartips according to the present disclosure may be manufactured in several single sizes. Notably, any of the features of any of the eartips described in the present disclosure may be combined and/or interchanged with any other eartips described in the present disclosure. Furthermore, the shape, size, features and/or functionality of any of the components of the earphone device and/or hearbud housing device described in the present disclosure may be modified for each particular user for the shape and size of each user's ear aperture and/or ear canal, or a combination thereof.

Notably, in experiments conducted using the eartip, the experiments have shown that the eartip allows for similar levels of sound isolation when compared to conventional foam and/or flange tips. For example, experiments have shown that the eartips provided in the present disclosure provided a NRR of 18 with a generally flat high frequency profile. A flat attenuation profile maintains an ambient environment's frequency profile when level reduced by the attenuation, which can be useful in maintaining the quality of ambient speech and music (or other audio content) during the level reduction process.

In further embodiments, the eartip may be configured to have an open configuration prior to insertion onto a stent of the earphone housing and/or the earphone device 2415 itself. By having an open configuration, the eartip may be mass produced using conventional molding techniques and/or by utilizing 3D commercial printers. The open configuration of the eartip also facilitates molding, and can be 3D printed, where the open configuration allows for resin removal. For example, resin removal may be achieved by utilizing commercial 3D printers that allow the use of lower durometer materials, such as Stratasys machines and the like. In certain embodiments, since the eartip has an open configuration, which is then sealed, any additional pressure can force encapsulated gas out of the eartip relieving the feedback pressure so as to keep the comfort level for the first user 2401 relatively stable.

In addition to the first user 2401, the system 2400 may include a second user 2420, who may utilize a fourth user device 2421 to access data, content, and applications, or to perform a variety of other tasks and functions. Much like the first user 2401, the second user 2420 may be may be any type of user that may potentially desire to listen to audio content, such as from, but not limited to, a storage device of the fourth user device 2421, a telephone call that the second user 2420 is participating in, audio content occurring in an environment in proximity to the second user 2420, any other type of audio content, or a combination thereof. For example, the second user 2420 may be an individual that may be listening to songs stored in a playlist that resides on the fourth user device 2421. Also, much like the first user 2401, the second user 2420 may utilize fourth user device 2421 to access an application (e.g., a browser or a mobile application) executing on the fourth user device 2421 that may be utilized to access web pages, data, and content associated with the system 2400. The fourth user device 2421 may include a memory 2422 that includes instructions, and a processor 2423 that executes the instructions from the memory 2422 to perform the various operations that are performed by the fourth user device 2421. In certain embodiments, the processor 2423 may be hardware, software, or a combination thereof. The fourth user device 2421 may also include an interface 2424 (e.g., a screen, a monitor, a graphical user interface, etc.) that may enable the second user 2420 to interact with various applications executing on the fourth user device 2421, to interact with various applications executing in the system 2400, and to interact with the system 2400. In certain embodiments, the fourth user device 2421 may include any number of transducers, such as, but not limited to, microphones, speakers, any type of audio-based transducer, any type of transducer, or a combination thereof. In certain embodiments, the fourth user device 2421 may be a computer, a laptop, a tablet device, a phablet, a server, a mobile device, a smartphone, a smart watch, and/or any other type of computing device. Illustratively, the fourth user device 2421 may be a computing device in FIG. 9. The fourth user device 2421 may also include any of the componentry described for first user device 2402, the second user device 2406, and/or the third user device 2410. In certain embodiments, the fourth user device 2421 may also include a global positioning system (GPS), which may include a GPS receiver and any other necessary components for enabling GPS functionality, accelerometers, gyroscopes, sensors, and any other componentry suitable for a computing device.

In addition to using fourth user device 2421, the second user 2420 may also utilize and/or have access to a fifth user device 2425. As with fourth user device 2421, the second user 2420 may utilize the fourth and fifth user devices 2421, 2425 to transmit signals to access various online services and content. The fifth user device 2425 may include a memory 2426 that includes instructions, and a processor 2427 that executes the instructions from the memory 2426 to perform the various operations that are performed by the fifth user device 2425. In certain embodiments, the processor 2427 may be hardware, software, or a combination thereof. The fifth user device 2425 may also include an interface 2428 that may enable the second user 2420 to interact with various applications executing on the fifth user device 2425 and to interact with the system 2400. In certain embodiments, the fifth user device 2425 may include any number of transducers, such as, but not limited to, microphones, speakers, any type of audio-based transducer, any type of transducer, or a combination thereof. In certain embodiments, the fifth user device 2425 may be and/or may include a computer, any type of sensor, a laptop, a set-top-box, a tablet device, a phablet, a server, a mobile device, a smartphone, a smart watch, and/or any other type of computing device. Illustratively, the fifth user device 2425 is shown as a tablet device in FIG. 15.

The fourth and fifth user devices 2421, 2425 may belong to and/or form a communications network 2431. In certain embodiments, the communications network 2431 may be a local, mesh, or other network that facilitates communications between the fourth and fifth user devices 2421, 2425, and/or any other devices, programs, and/or networks of system 2400 or outside system 2400. In certain embodiments, the communications network 2431 may be formed between the fourth and fifth user devices 2421, 2425 through the use of any type of wireless or other protocol and/or technology. For example, the fourth and fifth user devices 2421, 2425 may communicate with one another in the communications network 2416, such as by utilizing BLE, classic Bluetooth, ZigBee, cellular, NFC, Wi-Fi, Z-Wave, ANT+, IEEE 802.15.4, IEEE 802.22, ISA100a, infrared, ISM band, RFID, UWB, Wireless HD, Wireless USB, any other protocol and/or wireless technology, satellite, fiber, or any combination thereof. Notably, the communications network 2431 may be configured to communicatively link with and/or communicate with any other network of the system 2400 and/or outside the system 2400.

Much like first user 2401, the second user 2420 may have his or her own earphone device 2430. The earphone device 2430 may be utilized by the second user 2420 to hear and/or audition audio content, transmit audio content, receive audio content, experience any type of content, process audio content, adjust audio content, store audio content, perform any type of operation with respect to audio content, or a combination thereof. The earphone device 2430 may be an earpiece, a hearing aid, an ear monitor, an ear terminal, a behind-the-ear device, any type of acoustic device, or a combination thereof. The earphone device 2430 may include any type of component utilized for any type of earpiece, and may include any of the features, functionality and/or components described and/or usable with earphone device 2415. For example, earphone device 2430 may include any number of transceivers, ear canal microphones, ambient sound microphones, processors, memories, housings, eartips, foam tips, flanges, any other component, or any combination thereof.

In certain embodiments, the first, second, third, fourth, and/or fifth user devices 2402, 2406, 2410, 2421, 2425 and/or earphone devices 2415, 2430 may have any number of software applications and/or application services stored and/or accessible thereon. For example, the first and second user devices 2402, 2411 may include applications for processing audio content, applications for playing, editing, transmitting, and/or receiving audio content, streaming media applications, speech-to-text translation applications, cloud-based applications, search engine applications, natural language processing applications, database applications, algorithmic applications, phone-based applications, product-ordering applications, business applications, e-commerce applications, media streaming applications, content-based applications, database applications, gaming applications, internet-based applications, browser applications, mobile applications, service-based applications, productivity applications, video applications, music applications, social media applications, presentation applications, any other type of applications, any types of application services, or a combination thereof. In certain embodiments, the software applications and services may include one or more graphical user interfaces so as to enable the first and second users 2401, 2420 to readily interact with the software applications. The software applications and services may also be utilized by the first and second users 2401, 2420 to interact with any device in the system 2400, any network in the system 2400 (e.g. communications networks 2416, 2431, 2435), or any combination thereof. For example, the software applications executing on the first, second, third, fourth, and/or fifth user devices 2402, 2406, 2410, 2421, 2425 and/or earphone devices 2415, 2430 may be applications for receiving data, applications for storing data, applications for auditioning, editing, storing and/or processing audio content, applications for receiving demographic and preference information, applications for transforming data, applications for executing mathematical algorithms, applications for generating and transmitting electronic messages, applications for generating and transmitting various types of content, any other type of applications, or a combination thereof. In certain embodiments, the first, second, third, fourth, and/or fifth user devices 2402, 2406, 2410, 2421, 2425 and/or earphone devices 2415, 2430 may include associated telephone numbers, internet protocol addresses, device identities, or any other identifiers to uniquely identify the first, second, third, fourth, and/or fifth user devices 2402, 2406, 2410, 2421, 2425 and/or earphone devices 2415, 2430 and/or the first and second users 2401, 2420. In certain embodiments, location information corresponding to the first, second, third, fourth, and/or fifth user devices 2402, 2406, 2410, 2421, 2425 and/or earphone devices 2415, 2430 may be obtained based on the internet protocol addresses, by receiving a signal from the first, second, third, fourth, and/or fifth user devices 2402, 2406, 2410, 2421, 2425 and/or earphone devices 2415, 2430 or based on profile information corresponding to the first, second, third, fourth, and/or fifth user devices 2402, 2406, 2410, 2421, 2425 and/or earphone devices 2415, 2430.

The system 2400 may also include a communications network 2435. The communications network 2435 may be under the control of a service provider, the first and/or second users 2401, 2420, any other designated user, or a combination thereof. The communications network 2435 of the system 2400 may be configured to link each of the devices in the system 2400 to one another. For example, the communications network 2435 may be utilized by the first user device 2402 to connect with other devices within or outside communications network 2435. Additionally, the communications network 2435 may be configured to transmit, generate, and receive any information and data traversing the system 2400. In certain embodiments, the communications network 2435 may include any number of servers, databases, or other componentry. The communications network 2435 may also include and be connected to a mesh network, a local network, a cloud-computing network, an IMS network, a VoIP network, a security network, a VoLTE network, a wireless network, an Ethernet network, a satellite network, a broadband network, a cellular network, a private network, a cable network, the Internet, an internet protocol network, MPLS network, a content distribution network, any network, or any combination thereof. Illustratively, servers 2440, 2445, and 2450 are shown as being included within communications network 2435. In certain embodiments, the communications network 2435 may be part of a single autonomous system that is located in a particular geographic region or be part of multiple autonomous systems that span several geographic regions.

Notably, the functionality of the system 2400 may be supported and executed by using any combination of the servers 2440, 2445, 2450, and 2460. The servers 2440, 2445, and 2450 may reside in communications network 2435, however, in certain embodiments, the servers 2440, 2445, 2450 may reside outside communications network 2435. The servers 2440, 2445, and 2450 may provide and serve as a server service that performs the various operations and functions provided by the system 2400. In certain embodiments, the server 2440 may include a memory 2441 that includes instructions, and a processor 2442 that executes the instructions from the memory 2441 to perform various operations that are performed by the server 2440. The processor 2442 may be hardware, software, or a combination thereof. Similarly, the server 2445 may include a memory 2446 that includes instructions, and a processor 2447 that executes the instructions from the memory 2446 to perform the various operations that are performed by the server 2445. Furthermore, the server 2450 may include a memory 2451 that includes instructions, and a processor 2452 that executes the instructions from the memory 2451 to perform the various operations that are performed by the server 2450. In certain embodiments, the servers 2440, 2445, 2450, and 2460 may be network servers, routers, gateways, switches, media distribution hubs, signal transfer points, service control points, service switching points, firewalls, routers, edge devices, nodes, computers, mobile devices, or any other suitable computing device, or any combination thereof. In certain embodiments, the servers 2440, 2445, 2450 may be communicatively linked to the communications network 2435, the communications network 2416, the communications network 2431, any network, any device in the system 2400, any program in the system 2400, or any combination thereof.

The database 2455 of the system 2400 may be utilized to store and relay information that traverses the system 2400, cache content that traverses the system 2400, store data about each of the devices in the system 2400 and perform any other typical functions of a database. In certain embodiments, the database 2455 may be connected to or reside within the communications network 2435, the communications network 2416, the communications network 2431, any other network, or a combination thereof. In certain embodiments, the database 2455 may serve as a central repository for any information associated with any of the devices and information associated with the system 2400. Furthermore, the database 2455 may include a processor and memory or be connected to a processor and memory to perform the various operation associated with the database 2455. In certain embodiments, the database 2455 may be connected to the earphone devices 2415, 2430, the servers 2440, 2445, 2450, 2460, the first user device 2402, the second user device 2406, the third user device 2410, the fourth user device 2421, the fifth user device 2425, any devices in the system 2400, any other device, any network, or any combination thereof.

The database 2455 may also store information and metadata obtained from the system 2400, store metadata and other information associated with the first and second users 2401, 2420, store user profiles associated with the first and second users 2401, 2420, store device profiles associated with any device in the system 2400, store communications traversing the system 2400, store user preferences, store information associated with any device or signal in the system 2400, store information relating to patterns of usage relating to the first, second, third, fourth, and fifth user devices 2402, 2406, 2410, 2421, 2425, store audio content associated with the first, second, third, fourth, and fifth user devices 2402, 2406, 2410, 2421, 2425 and/or earphone devices 2415, 2430, store audio content and/or information associated with the audio content that is captured by the ambient sound microphones, store audio content and/or information associated with audio content that is captured by ear canal microphones, store any information obtained from any of the networks in the system 2400, store audio content and/or information associated with audio content that is outputted by ear canal receivers of the system 2400, store any information and/or signals transmitted and/or received by transceivers of the system 2400, store any device and/or capability specifications relating to the earphone devices 2415, 2430, store historical data associated with the first and second users 2401, 2415, store information relating to the size (e.g. depth, height, width, curvatures, etc.) and/or shape of the first and/or second user's 2401, 2420 ear canals and/or ears, store information identifying and or describing any eartip utilized with the earphone devices 2401, 2415, store device characteristics for any of the devices in the system 2400, store information relating to any devices associated with the first and second users 2401, 2420, store any information associated with the earphone devices 2415, 2430, store log on sequences and/or authentication information for accessing any of the devices of the system 2400, store information associated with the communications networks 2416, 2431, store any information generated and/or processed by the system 2400, store any of the information disclosed for any of the operations and functions disclosed for the system 2400 herewith, store any information traversing the system 2400, or any combination thereof. Furthermore, the database 2455 may be configured to process queries sent to it by any device in the system 2400.

The system 2400 may also include a software application, which may be configured to perform and support the operative functions of the system 2400, such as the operative functions of the first, second, third, fourth, and fifth user devices 2402, 2406, 2410, 2421, 2425 and/or the earphone devices 2415, 2430. In certain embodiments, the application may be a website, a mobile application, a software application, or a combination thereof, which may be made accessible to users utilizing one or more computing devices, such as the first, second, third, fourth, and fifth user devices 2402, 2406, 2410, 2421, 2425 and/or the earphone devices 2415, 2430. The application of the system 2400 may be accessible via an internet connection established with a browser program or other application executing on the first, second, third, fourth, and fifth user devices 2402, 2406, 2410, 2421, 2425 and/or the earphone devices 2415, 2430, a mobile application executing on the first, second, third, fourth, and fifth user devices 2402, 2406, 2410, 2421, 2425 and/or the earphone devices 2415, 2430, or through other suitable means. Additionally, the application may allow users and computing devices to create accounts with the application and sign-in to the created accounts with authenticating username and password log-in combinations. The application may include a custom graphical user interface that the first user 2401 or second user 2420 may interact with by utilizing a browser executing on the first, second, third, fourth, and fifth user devices 2402, 2406, 2410, 2421, 2425 and/or the earphone devices 2415, 2430. In certain embodiments, the software application may execute directly as an installed program on the first, second, third, fourth, and fifth user devices 2402, 2406, 2410, 2421, 2425 and/or the earphone devices 2415, 2430.

### Computing System for Facilitating the Operation and Functionality of the System

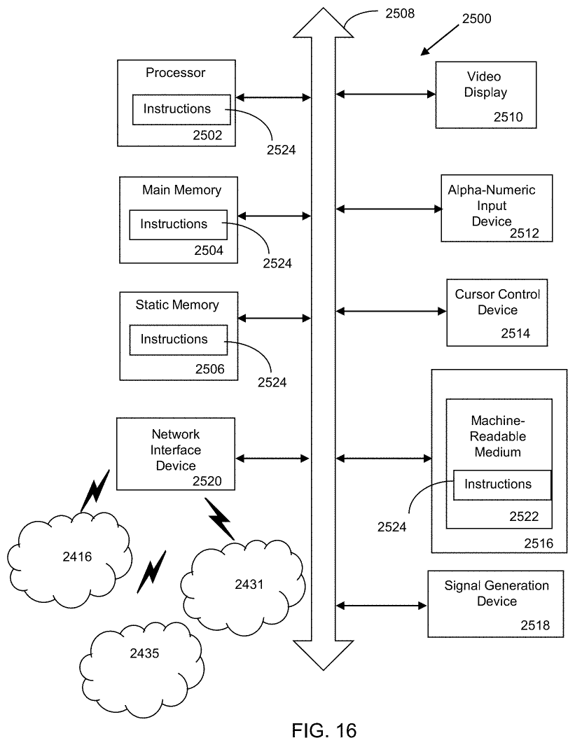

Referring now also to FIG. 16, at least a portion of the methodologies and techniques described with respect to the exemplary embodiments of the system 2400 can incorporate a machine, such as, but not limited to, computer system 2500, or other computing device within which a set of instructions, when executed, may cause the machine to perform any one or more of the methodologies or functions discussed above. The machine may be configured to facilitate various operations conducted by the system 2400. For example, the machine may be configured to, but is not limited to, assist the system 2400 by providing processing power to assist with processing loads experienced in the system 2400, by providing storage capacity for storing instructions or data traversing the system 2400, by providing functionality and/or programs for facilitating the operative functionality of the earphone devices 2415, 2430, and/or the first, second, third, fourth, and fifth user devices 2402, 2406, 2410, 2421, 2425 and/or the earphone devices 2415, 2430, by providing functionality and/or programs for facilitating operation of any of the components of the earphone devices 2415, 2430 (e.g. ear canal receivers, transceivers, ear canal microphones, ambient sound microphones, or by assisting with any other operations conducted by or within the system 2400.

In some embodiments, the machine may operate as a standalone device. In some embodiments, the machine may be connected (e.g., using communications network 2435, the communications network 2416, the communications network 2431, another network, or a combination thereof) to and assist with operations performed by other machines and systems, such as, but not limited to, the first user device 2402, the second user device 2411, the third user device 2410, the fourth user device 2421, the fifth user device 2425, the earphone device 2415, the earphone device 2430, the server 2440, the server 2450, the database 2455, the server 2460, or any combination thereof. The machine may be connected with any component in the system 2400. In a networked deployment, the machine may operate in the capacity of a server or a client user machine in a server-client user network environment, or as a peer machine in a peer-to-peer (or distributed) network environment. The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computer system 2500 may include a processor 2502 (e.g., a central processing unit (CPU), a graphics processing unit (GPU), a main memory 2504 and a static memory 2506, which communicate with each other via a bus 2508. The computer system 2500 may further include a video display unit 2510, which may be, but is not limited to, a liquid crystal display (LCD), a flat panel, a solid state display, or a cathode ray tube (CRT). The computer system 2500 may include an input device 2512, such as, but not limited to, a keyboard, a cursor control device 2514, such as, but not limited to, a mouse, a disk drive unit 2516, a signal generation device 2518, such as, but not limited to, a speaker or remote control, and a network interface device 2520.

The disk drive unit 2516 may include a machine-readable medium 2522 on which is stored one or more sets of instructions 2524, such as, but not limited to, software embodying any one or more of the methodologies or functions described herein, including those methods illustrated above. The instructions 2524 may also reside, completely or at least partially, within the main memory 2504, the static memory 2506, or within the processor 2502, or a combination thereof, during execution thereof by the computer system 2500. The main memory 2504 and the processor 2502 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine-readable medium 2522 containing instructions 2524 so that a device connected to the communications network 2435, the communications network 2416, the communications network 2431, another network, or a combination thereof, can send or receive voice, video or data, and communicate over the communications network 2435, the communications network 2416, the communications network 2431, another network, or a combination thereof, using the instructions. The instructions 2524 may further be transmitted or received over the communications network 2435, another network, or a combination thereof, via the network interface device 2520.

While the machine-readable medium 2522 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure.

The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain embodiments, may not include a wave or signal per se. Accordingly, the disclosure is considered to include any one or more of a machine-readable medium or a distribution medium, as listed herein and including art-recognized equivalents and successor media, in which the software implementations herein are stored.

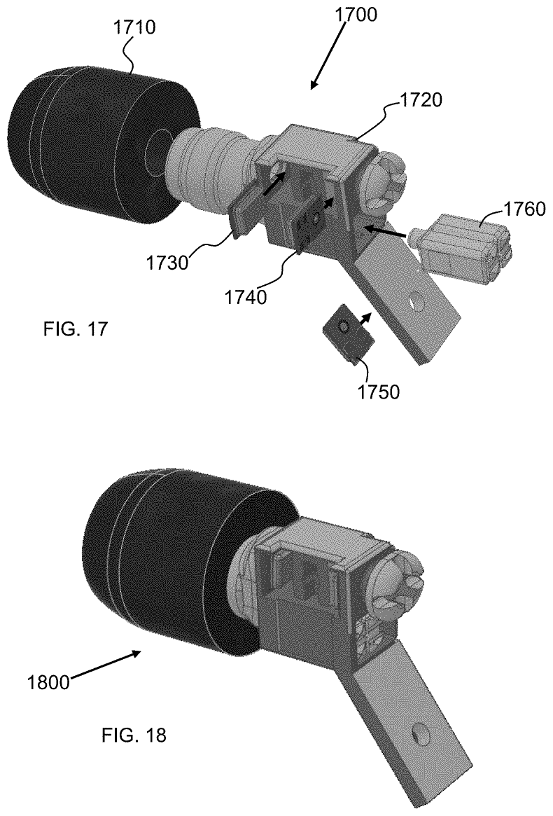

FIG. 17 is a schematic exploded view of a multimicrophone earphone 1700. The earphone 1700 can include an eartip 1710, an electronic package housing 1720, a first microphone 1730 (e.g., an ECM), a second microphone 1740 (e.g., a first ASM), a third microphone 1740 (e.g. a second ASM), and a speaker 1760, where FIG. 18 illustrates the earphone 1800 of FIG. 17 with components inserted.

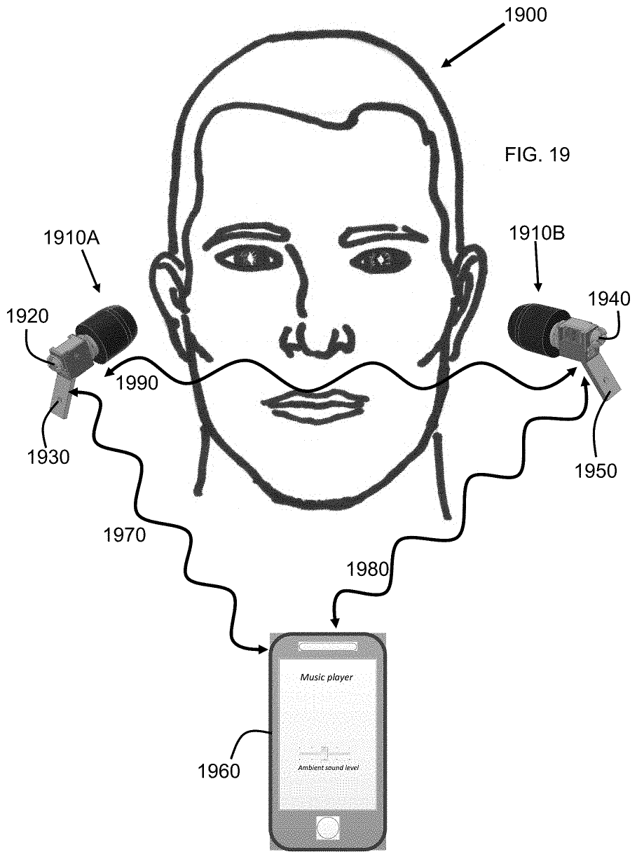

FIG. 19 illustrates left 1910A and right earphones 1910B prior to insertion of a user's ears. The Earphones can have a total of at least four ASMs (1920, 1930, 1940, and 1950), which can be used in accordance with the description herein for example beam forming and voice detection and/or identification. For example, FIG. 2B illustrates the use of three ASMs. Two of these can be in earphone 1910A while the remainder is in earphone 1910B or vice versa. Additionally, all four or more can be used. FIG. 19 additionally shows a communication device which can include an ASM. Thus, each earphone (1910A, 1910B) can each contribute an ASM and the communication device 1970 the third ASM or all ASMs of communicatively coupled (e.g, 1970, 1980, 1990) devices (earphones, communication device 1960 (e.g., phone, ipad, computer) can be used.

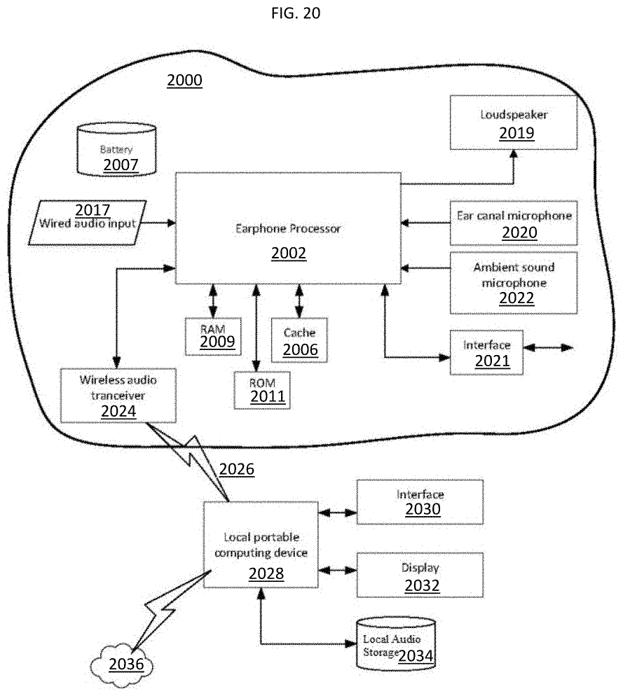

FIG. 20 is a block diagram of an electronic earphone device suitable for use with at least one of the described embodiments. The electronic device 2000 illustrates circuitry of a representative computing device. The electronic device 2000 includes a processor 2002 that pertains to a Digital Signal Processor (DSP) device or microprocessor or controller for controlling the overall operation of the electronic device 2000. For example, processor 2002 can be used to receive a wireless 2024 or wired 2017 audio input signals. The electronic device 2000 can also include a cache 2006. The cache 2006 is, for example, Random Access Memory (RAM) provided by semiconductor memory. The relative access time to the cache 2006 is substantially shorter than for the system RAM 2009.

The electronic device 2000 is powered by a battery 2007. The electronic device 2000 (e.g., earphone) can also include the RAM 2009 and a Read-Only Memory (ROM) 2011. The ROM 2011 can store programs, utilities or processes to be executed in a non-volatile manner.

The speaker 2019 is an ear canal loudspeaker, also often referred to as a receiver. Microphone 2020 can be used to detect audible sound in the ear canal (ear canal microphone). A second microphone 2022 can be used to detect audible sound in the ambient environment (ambient sound microphone).

An optional interface 2021 on the earphone device 2000 can be used for user input, such as a capacitive touch sensor.

A wireless audio and data transceiver unit 2024 connects with a computing device 2028 (e.g., a local portable computing device). The wireless connection 2026 can be any electromagnetic connection, for example via Bluetooth or Wifi or magnetic induction, and transmits audio and control data. The local portable computing device 2028 can be a mobile phone, tablet, television, gaming hardware unit or other similar hardware devices.

The local portable computing device 2028 utilizes a user interface 2030 and display 2032, such as a touch screen or buttons, and can be connected to the cloud 2036 to receive and stream audio. Alternatively, audio can be replayed to the earphone device 2000 from storage 2034 on the computing device 2028.

Although only one processor is illustrated for an information processing system, information processing systems with multiple CPUs or processors can be used equally effectively. Various embodiments of the present disclosure can further incorporate interfaces that each includes separate, fully programmed microprocessors that are used to off-load processing from the processor. An operating system (not shown) included in main memory for the information processing system may be a suitable multitasking and/or multiprocessing operating system, such as, but not limited to, any of the iOS, Linux, UNIX, Windows, and Windows Server based operating systems. Various embodiments of the present disclosure are able to use any other suitable operating system. Various embodiments of the present disclosure utilize architectures, such as an object oriented framework mechanism, that allows instructions of the components of operating system (not shown) to be executed on any processor located within the information processing system. Various embodiments of the present disclosure are able to be adapted to work with any data communications connections including present day analog and/or digital techniques or via a future networking mechanism.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The term “another”, as used herein, is defined as at least a second or more. The terms “including” and “having,” as used herein, are defined as comprising (i.e., open language). The term “coupled,” as used herein, is defined as “connected,” although not necessarily directly, and not necessarily mechanically. “Communicatively coupled” refers to coupling of components such that these components are able to communicate with one another through, for example, wired, wireless or other communications media. The terms “communicatively coupled” or “communicatively coupling” include, but are not limited to, communicating electronic control signals by which one element may direct or control another. The term “configured to” describes hardware, software or a combination of hardware and software that is adapted to, set up, arranged, built, composed, constructed, designed or that has any combination of these characteristics to carry out a given function. The term “adapted to” describes hardware, software or a combination of hardware and software that is capable of, able to accommodate, to make, or that is suitable to carry out a given function.

The terms “controller”, “computer”, “processor”, “server”, “client”, “computer system”, “computing system”, “personal computing system”, “processing system”, or “information processing system”, describe examples of a suitably configured processing system adapted to implement one or more embodiments herein. Any suitably configured processing system is similarly able to be used by embodiments herein, for example and not for limitation, a personal computer, a laptop personal computer (laptop PC), a tablet computer, a smart phone, a mobile phone, a wireless communication device, a personal digital assistant, a workstation, and the like. A processing system may include one or more processing systems or processors. A processing system can be realized in a centralized fashion in one processing system or in a distributed fashion where different elements are spread across several interconnected processing systems.

The corresponding structures, materials, acts, and equivalents of all means or step plus function elements in the claims below are intended to include any structure, material, or act for performing the function in combination with other claimed elements as specifically claimed. The description herein has been presented for purposes of illustration and description, but is not intended to be exhaustive or limited to the examples in the form disclosed. Many modifications and variations will be apparent to those of ordinary skill in the art without departing from the scope of the examples presented or claimed. The disclosed embodiments were chosen and described in order to explain the principles of the embodiments and the practical application, and to enable others of ordinary skill in the art to understand the various embodiments with various modifications as are suited to the particular use contemplated. It is intended that the appended claims below cover any and all such applications, modifications, and variations within the scope of the embodiments.

What is claimed is:

1. An earpiece comprising:
a speaker;
an ear canal microphone;
an ambient sound microphone;
a stent configured to accept an eartip;
a contraction/expansion first element;
a contraction/expansion second element, where the first and second elements are embedded in the stent;
a memory that stores instructions; and
a processor that is configured to execute the instructions to perform operations, the operations comprising:
sending a signal to the first element so that the first element contracts or expands bending the stent.

2. The earpiece according to claim 1 wherein the operations further include:
sending a signal to the second element so that the second element contracts or expands bending the stent.

3. The earpiece according to claim 2 further comprising:
a sensor coupled to the stent, wherein the sensor is configured to determine the distance of a stent from a wall of a tubular structure when the stent is inserted into the tubular structure.

4. The earpiece according to claim 3, wherein the tubular structure is an ear canal of a user.

5. The earpiece according to claim 4, wherein the operations further include:
receiving a microphone signal.

6. The earpiece according to claim 5, wherein the operations further include:
sending an audio signal to the speaker.

7. The earpiece according to claim 6, further comprising:
a second ambient sound microphone.

8. The earpiece according to claim 7, where the first element is composed of twisted polymer whose expansion and contraction is controlled by heating.

9. The earpiece according to claim 8, wherein the first element is further composed of twisted conductive thread.

10. The earpiece according to claim 9 wherein the first element's expansion and contraction are controlled by the processor sending current through the twisted conductive thread.

11. The earpiece according to claim 7, wherein the eartip seals the ear canal when the earpiece is inserted into the ear canal.

12. The earpiece according to claim 7, wherein the sensor is an infrared sensor.

13. The earpiece according to claim 7, wherein the sensor is an acoustic sensor.
